(19) United States
(12) Patent Application Publication
(10) Pub. No.: US 2023/0346650 A1
(43) Pub. Date: Nov. 2, 2023

(54) POWDER-BASED NAIL POLISH AND METHODS OF MANUFACTURING AND APPLYING THE SAME

(71) Applicant: Teri Lea Zaite, Loxahatchee, FL (US)

(72) Inventor: Teri Lea Zaite, Loxahatchee, FL (US)

(21) Appl. No.: 18/346,356

(22) Filed: Jul. 3, 2023

Related U.S. Application Data
(63) Continuation-in-part of application No. 16/670,089, filed on Oct. 31, 2019.
Application No. 16/670,089 is a continuation of application No. 15/725,565, filed on Oct. 5, 2017, now abandoned.
(60) Provisional application No. 62/411,395, filed on Oct. 21, 2016.

(51) Int. Cl. A61K 8/02; A61Q 3/02; A61K 8/26; A61K 8/36; A61K 8/64; A61K 8/67; A45D 29/12; A61K 8/98

(52) U.S. Cl. CPC A61K 8/022; A61Q 3/02; A61K 8/26; A61K 8/361; A61K 8/64; A61K 8/678; A45D 29/12; A61K 8/987; A61K 2800/43; A61K 2800/805; A61K 2800/30; A61K 2800/87; A61K 2800/81; A45D 33/006

(57) ABSTRACT

A dry powder-based nail polish composition is disclosed. The composition includes a plurality of ingredients including predetermined weighed amounts of kaolin; sericite mica (uncoated and coated with carnauba wax); magnesium myristate; micro fine silk powder; delaminated talc; and mixed tocopherols, with each ingredient being present as a powder. Also disclosed are methods of applying powder-based nail polish compositions to nails of people and of manufacturing the nail polish composition from select powder ingredients.

### REFERENCE TO RELATED APPLICATIONS

The present application is a continuation-in-part (“CIP”) application, based upon U.S. Ser. No. 16/670,089 (filed Oct. 31, 2019) which is a continuation of U.S. Ser. No. 15/725,565 (filed Oct. 5, 2017) which is based on U.S. provisional application No. 62/411,395 (filed Oct. 21, 2016), all of which are hereby incorporated by reference in their entirety for priority purposes pursuant to Title 35, United States Code, Section 120.

### FIELD

The present subject matter is directed in general to nail polish compositions and more particularly is directed to a customizable powder-based nail polish and to methods of custom-manufacturing the powder-based nail polish and of coating nails with the same.

### BACKGROUND

Current methods of applying liquid nail polishes can be frustrating and difficult to complete in a reasonable amount of time when detailed work is required and may even result in a mess when a polish-applying hand cramps up or a non-dominant hand is used.

U.S. Pat. Nos. 4,626,428 and 4,669,491 (both to Weisberg et al.) each disclose a process for applying a protective acrylic coating to human fingernails. Both processes include these six steps: (1) cleaning (or clearing) and roughening the surface of a nail; (2) applying (2a) a slow-curing cyanoacrylate glue or (2b) a layer of liquid acrylic monomer; (3) applying a powdered polymethacrylate ester to the wetted nail surface; (4) removing loose powder and smoothing a nail surface; (5) brushing on, as one would with nail polish, a blend of acrylic monomers; after curing, (6) finishing the nail in a conventional manner.

U.S. Pat. No. 4,798,720 to Holder discloses a fingernail-treating composition prepared by mixing a topcoat nail polish, an acrylic nail powder, acrylic nail primer, and an adhesive.

U.S. Pat. No. 5,747,018 to Valenty discloses thickening agents, said to keep fine powders in suspension, to provide uniform colloidal mixtures for application to fingernails.

U.S. Pat. No. 5,830,442 to Beaver discloses a pigmented artificial acrylic fingernail made from a liquid methacrylate monomer, a powdered acrylic or methacrylate polymer, and a catalyst including an activator and an accelerator. A pigment is mixed with the powder so that the resultant nail has the glossy, opaque appearance expected from a conventionally applied nail polish. The pigment is present in the range of 1.5-15% by weight of the powder and exhibits a color having a wavelength of 4000-7000 Angstroms.

US publication 2003/0082121 to Borsakian et al. discloses a color-changing nail polish composition including a nail polish base into which a first colorant, a second colorant, and a UV photochromic powder are mixed. The photochromic powder together with the first and second colorants are said to impart—after the nail polish composition is applied to a fingernail of a human and allowed to dry— a first color when the nail is at normal body temperature, a second color when the nail temperature is above the normal body temperature, and a third color when the nail polish composition is exposed to UV radiation. US publication 2012/0308497 to Lee discloses a nail polish said to possess a crack and color changing effect. The nail polish includes a polymer, a solvent, a plasticizer, a pigment, a silica, and a thermochromic powder. The polymer accounts for 10%-30% of the total weight and is said to contain nitrocellulose and resin. The solvent accounts for 44%-64% of the total weight. The plasticizer accounts for 3%-8% of the total weight. Silica is said to account for 8%, and the pigment is said to account for 10%-20% of the total weight. Thermochromic powder is said to account for 5% of the total weight.

US publication 2014/0076342 to Huynh discloses an apparatus, a kit, and a method for applying a French manicure to a user's own nails or those of a customer. The apparatus includes a finger guide removably associated with an acrylic nail polish powder-containing vessel. A method for applying a French manicure using the apparatus is disclosed. The kit includes acrylic nail polish powder, a finger guide, and other materials, for causing the acrylic nail polish powder to become a solid nail polish coating.

Prior art described above does not solve a problem solved by the present subject matter. For instance, users who apply liquid nail polish frequently complain about getting the polish on the cuticles (or sides of fingers), which must be removed by polish remover.

Not only does having to remove the mess of excess nail polish involve additional steps, but frequently the excess nail polish results in a difficult to remove stain. Users of such nail polish methods will need to wait for the polish to dry, which often leads to nicks and smudges. Currently, so-called “dip” nail polishes systems require users to dip their entire finger into a polymeric powder including pigment, which may lead to spills of the powder or pigment, or stains on fingers from the pigment. Current dip nail polish systems also use a buffer or grinder to remove excess, which can damage one's natural nail bed.

It would therefore be appreciated by the public if: (1) customizable powder-based fingernail polish; (2) methods for custom-manufacturing powder-based fingernail polish; and (3) methods for coating nails with powder fingernail polish compositions are available.

As can also be appreciated, there is a desire—by people wishing to “customize” their nails—for an easy-to-use method of applying a truly unique nail polish composition, which reduces nail polish application time and minimizes clean-up time, by using a nail polish that is “powder dry” and requires only about 30 sec. after applying a topcoat to dry, e.g., under an LED (“light-emitting diode”) lamp or bulb. Accordingly, by brush-painting nails with a powder nail polish composition formulated to stick only to a base coat and not the rest of a finger or cuticle, the present subject matter overcomes disadvantages of the prior art, using a nail polish that is applied-to-dryness, in minutes, to reduce any likelihood of ruining the dry powder-based nail polish application. Being able to customize one's own nail polish application by combining selected colors truly empowers people who use the powder-based nail polish of the present subject matter. The powder-based nail polish composition of the present subject matter, e.g., enables a person applying the powder nail polish to oneself or another person to use a variety of different colors, if desired. For, instance, one could have red and green “custom” designs for holidays and/or yellow and blue “custom” designs for other purposes. Applying a powder of one color, to the sticky base coat, immediately adjacent to another color (previously applied to the sticky base coat) does not result in any “smearing” of the colors, since the later-applied color, being a powder, is easily brushed off underlying prior-applied color. Therefore, when using the nail polish composition of the present subject matter, a person is always free to customize nail surfaces with the colors of one's choice, which enables designing unique patterns, ombrés, geometric shapes, “custom” nail tips, and more, without waiting for colors to dry, and free from the anxiety resulting from smudging, smearing, and various other mistakes.

### SUMMARY

One aspect of the present subject matter is directed to a powdered fingernail polish composition, preferably “brush”-applied to fingernails of a person, includes a plurality of ingredients including but not limited to: a predetermined weighed amount of kaolin clay; a predetermined weighed amount of sericite mica (coated, uncoated); a predetermined weighed amount of magnesium myristate; a predetermined weighed amount of micro fine silk powder; a predetermined weighed amount of delaminated talc; and a predetermined weighed amount of mixed tocopherols, with each ingredient being present as a powder.

A method of manufacturing the powdered nail polish composition contemplates mixing the plural powder ingredients, via gentle tumbling, for predetermined time periods.

Another aspect of the present subject matter is directed to a method of applying the powder nail polish composition to a clear sticky or tacky base coat layer applied to the nails of a person. Accordingly, a preferred method of doing so includes providing a preselected brush applicator; loading the applicator with powder nail polish composition; and brushing powder nail polish composition onto a tacky base coat layer on each nail. In embodiments, a base coat or layer has a predetermined stickiness or tackiness value.

These and other aspects, features, and advantages of the present subject matter shall become better understood after reviewing the figures and the detailed description.

### BRIEF DESCRIPTION OF THE FIGURES

FIG. 1 depicts a method of producing a composition of the present subject matter.

FIG. 2 is a front elevational view of an exemplary applicator for the composition.

FIG. 3 is an exploded perspective view of applicator components shown in FIG. 2.

FIG. 4 presents a front elevational view of a presently preferred tumble blender.

FIG. 5 is a side elevational view of the tumble blender, from plane 5-5 in FIG. 4.

FIG. 6 is another side elevational view of the tumble blender presented in FIG. 4.

FIG. 7 presents an enlarged plan view within the tumble blender relative to FIG. 4.

### DETAILED DESCRIPTION

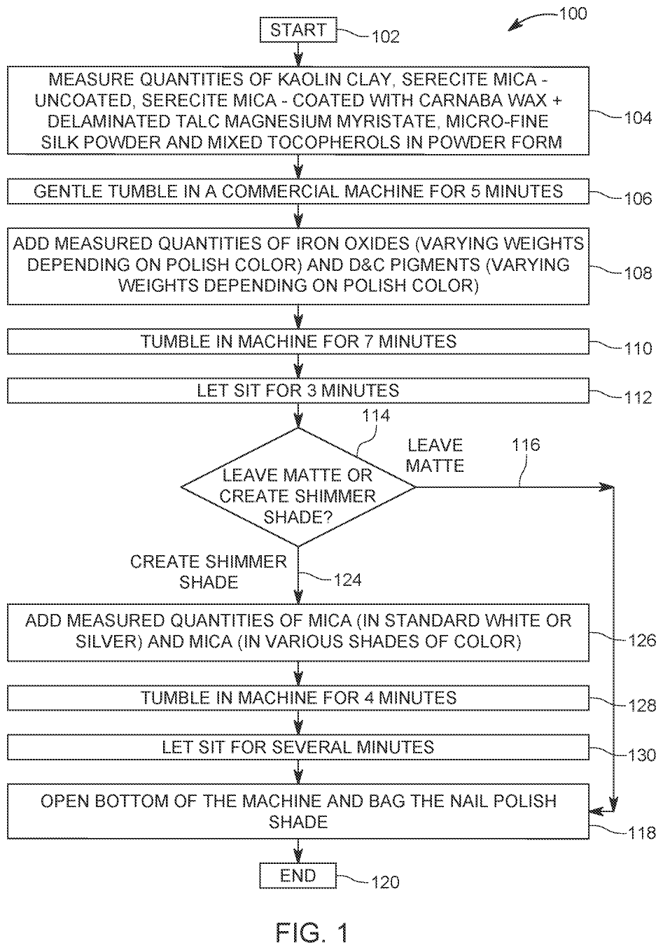

Referring initially to FIG. 1, an illustrative method 100 for producing a powdered nail polish composition of the present subject matter is initiated, or starts, at step 102, and thereafter proceeds to step 104, to measure predetermined weighed amounts of kaolin clay; sericite mica—uncoated; sericite mica—coated with carnauba wax; delaminated talc; magnesium myristate; micro-fine silk powder; and mixed tocopherols, all as powder.

Kaolin clay is a soft white clay. Kaolin is a hydrated aluminum silicate mineral that is characterized by its fine particle size, plate-like or lamellar particle shape, and chemical inertness. Kaolin is derived from the decomposition of certain other aluminous minerals, especially feldspars. Kaolin clay has cleansing and soothing effects on the skin and can also be used on teeth and hair and taken internally to manage diarrhea. Kaolin clay is found in soils around the world including China, the US, Brazil, and many other countries.

In embodiments, the amounts of kaolin, in relation to all powder-based nail polish composition ingredients, range from about 10 weight-percent (“wt.-%”) to about 35 wt.-%, or from about 10 weight-% to about 29 wt.-%, or from about 29 wt.-% to about 35 wt.-%.

Sericite is a name given to very fine, ragged grains and aggregates of white micas, typically composed of muscovite, illite, or paragonite. Sericite results from an alteration of orthoclase or plagioclase feldspars in regions that have been subjected to hydrothermal alteration, often associated with copper, tin, or other hydrothermal ore deposits. Sericite also occurs as a fine mica that gives a sheen to phyllite and schistose metamorphic rocks.

In embodiments, amounts of uncoated sericite mica, in relation to all ingredients in a powder-based nail polish composition, range from about 15 wt.-% to about 40 wt.-%, or from about 15 wt.-% to about 21.6 wt.-%, or from about 21.6 wt.-% to about 40 wt.-%.

Carnauba wax—having a melting point of about 85° C. (185° F.) and consisting primarily of esters of long-chain alcohols and acids— is a vegetable wax obtained from the fronds of the carnauba palm (Copernicia prunifera) of Brazil. Valued among natural waxes for its hardness and high melting temperature, carnauba wax is used as a vegan food-grade polish and as a hardening or gelling agent in a variety of commercial products.

In embodiments, the amounts of coated sericite mica, in relation to all powder nail polish composition ingredients, range from about 5 weight-percent (“wt.-%”) to about 15 wt.-%, from about 5 wt.-% to about 7.2 wt.-%, or from about 7.2 wt.-% to about 15 wt.-%.

Magnesium myristate—with chemical formula C₂₈H₅₄MgO₄ and molecular weight of 479.0 grams/mole—is the salt of magnesium and myristic acid (a fatty acid naturally occurring in palm, coconut oils). Magnesium myristate, existing only as a refined product, is a fine white powder having an approximate melting point of 130 to 150° C. (266 to 302° F.).

In embodiments, amounts of magnesium myristate relative to all powder nail polish composition ingredients, range from about 20 weight-percent (“wt.-%”) to about 50 wt.-%, or from about 20 wt.-% to about 32.5 wt.-%, or from about 32.5 wt.-% to about 50 wt.-%.

Micro fine silk powder—very fine, silky, and white— averages 5 microns in size. It will not dissolve in water. “Silk Powder” or “Powder Silk” is the full silk protein, composed entirely of Fibroin. Silk Powder is a yellow powder which, with a 200-mesh diameter, has the largest particle size and the heaviest molecular weight of three silk-based additives.

In embodiments, amounts of micro fine silk powder relative to all powder nail polish composition ingredients, range from about 1 weight-percent (“wt.-%”) to about 10 wt.-%, or from about 1 wt.-% to about 3.7 wt.-%, or from about 3.7 wt.-% to about 10 weight-%.

Talc, a clay mineral, is composed of hydrated magnesium silicate having chemical formula Mg₃Si₄O₁₀(OH)₂. Often occurring as “foliated” and/or “fibrous” clumps, talc also is occasionally found in exceptionally rare crystal form. Talc, with a perfect basal cleavage and an uneven flat fracture, is foliated with a two-dimensional platy form. The Mohs scale of mineral hardness, based on scratch hardness comparison, defines a value of 1 for talc “hardness,” the softest mineral. When scraped on a streak plate, talc produces a white streak. Talc, translucent to opaque, has a color ranging from whitish grey to green with a vitreous and pearly luster. Talc, insoluble in water, is slightly soluble in dilute mineral acids. Talc, a trioctahedral layered mineral, resembles pyrophyllite, but includes magnesium in the octahedral sites of the composite layers. The crystal structure of talc is described TOT, meaning that it is composed of parallel TOT layers weakly bonded to each other by weak van der Waals forces. The TOT layers, in turn, each consist of two tetrahedral sheets (T) strongly bonded to two faces of a single trioctahedral sheet (O). It is the weak bonding between TOT layers that gives talc its perfect basal cleavage and softness. The tetrahedral sheets consist of silica tetrahedra, which are silicon ions surrounded by four oxygen ions. The tetrahedra each share three of their four oxygen ions with neighboring tetrahedra to produce a hexagonal sheet. The remaining oxygen ion (the apical oxygen ion) is available to bond with the trioctahedral sheet. The trioctahedral sheet has the structure of a sheet of the mineral, brucite. The apical oxygens take the place of some of the hydroxyl ions that would be present in a brucite sheet, for tightly bonding tetrahedral sheets to the trioctahedral sheet. As their bulk composition is Si₄O₁₀⁴⁻, the tetrahedral sheets have a net negative charge; and as their bulk composition is Mg₃(OH)₂⁴⁺, the trioctahedral sheets have an equal positive charge. The combined TOT layer thus is electrically neutral. As hexagons in the T and O sheets are slightly different in size, the sheets are slightly distorted when they bond to a TOT layer. This breaks the hexagonal symmetry, reducing it to monoclinic or triclinic symmetry. However, the original hexahedral symmetry is discernible in the pseudo-trigonal character of talc crystals. Delamination is the separation of the layers of a laminar composite material, due to repeated stressing.

In embodiments, amounts of delaminated talc, in relation to all powder nail polish composition ingredients, range from about 3 weight-percent (“wt.-%”) to about 20 wt.-%, or from about 3 wt.-% to about 5.7 wt.-%, or from about 5.7 wt.-% to about 20 weight-%.

Tocopherols are a class of organic compounds comprising various methylated phenols, many of which have vitamin “E” activity. For instance, α-Tocopherol, a form of vitamin E, is preferentially absorbed and accumulated in humans, with measurement of vitamin E activity expressed in international units (“IU”). As there are three stereocenters in α-tocopherol, eight stereoisomers of α-tocopherol differ in their arrangement of groups around the three stereocenters. In RRR-α-tocopherol, all three stereocenters are in an “R” form. However, if a middle, of three stereocenters were changed (so hydrogen points down and a methyl group points up), the resultant structure becomes RSR-α-tocopherol. “Mixed tocopherols” contain at least 20% w/w other R, R, R-tocopherols (i.e., R, R, R-α-tocopherol content plus at least 25% R, R, R-β-; R, R, R-γ-; and R, R, R-δ-tocopherols).

While certain brands may contain 20.0% w/w or more of the several tocopherols noted above, including certain measurable tocotrienols, “mixed tocopherols” with higher γ-tocopherol content are marketed as “High Gamma-Tocopherol,” with labels reporting each component in milligrams, except R, R, R-α-tocopherol may be reported in “IU” units.

In embodiments, amounts of tocopherols (dry), in relation to all powder nail polish composition ingredients, range from about 0.1 weight-percent (“wt.-%”) to about 4 wt.-%, or from about 0.1 wt.-% to about 0.3 wt.-%, or from about 0.3 wt.-% to about 4 weight-%.

Referring again briefly to FIG. 1, weight-measured amounts of combined powder ingredients (kaolin; sericite mica, uncoated; sericite mica, coated with carnauba wax; delaminated talc; magnesium myristate; micro fine silk powder; and mixed tocopherols) are gently tumbled, at step 106, in a tumble blender for an amount of time ranging from about 1 minute (“min.”) to about 20 min., or from about 2 min. to about 15 min., or from about 3 min. to about 10 min., or from about 4 min. to about 7 min., or for about 5 minutes.

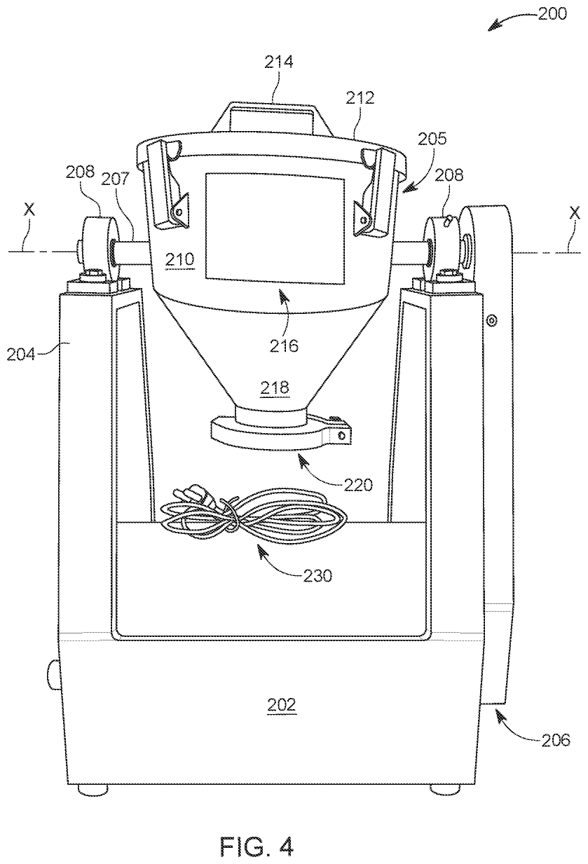

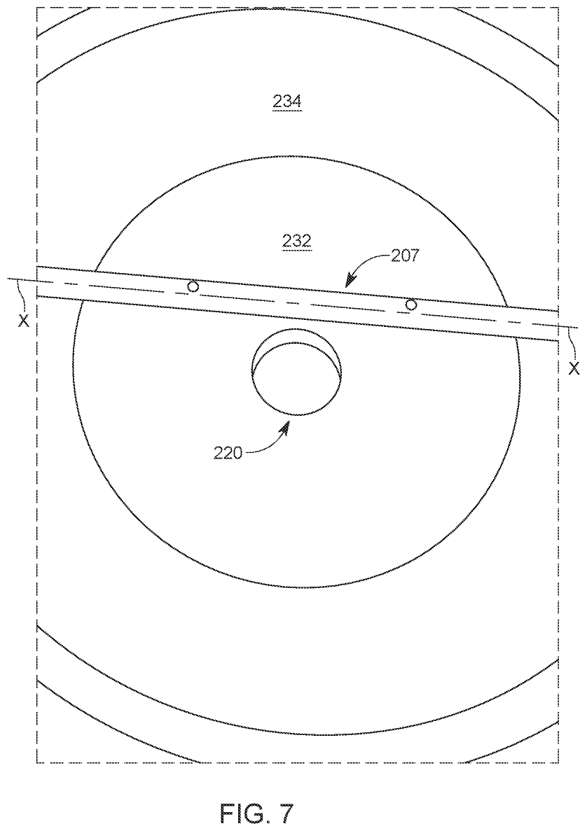

A preferred tumble blender 200 (FIGS. 4-6) includes a base 202, a spaced-apart pair of sides 204 unitary with the base 202, and a container 205 (FIG. 4) into which the weight-measured amounts of the combined powder nail polish ingredients are gently deposited. A cowling 206 (FIG. 4) is mounted on one of the sides 204. A drive mechanism (not shown) beneath the cowling 206 is used to rotate the container 205 mounted on a cylindrical member or rod 207 about a horizontally disposed axis X—X (FIGS. 4, 7). End portions of the cylindrical member or rod 207 are rotatably supported by a spaced-apart pair of pillow block bearings 208 secured atop respective spaced-apart blender sides 204.

The container 205 includes a hollow cylindrical upper section 210 (FIG. 4) having an inside surface 234 (FIG. 7), a hollow conical (or frustoconical) lower section 218 having an inside surface 232, with an outlet section 220 at the bottom of the lower section 218.

A cover 212 (having an integral handle 214) is supported atop the upper section 210. Instructions for operating the tumble blender 200, with safety considerations noted by enlarged colored fonts and a warning symbol, are secured to the upper section 210.

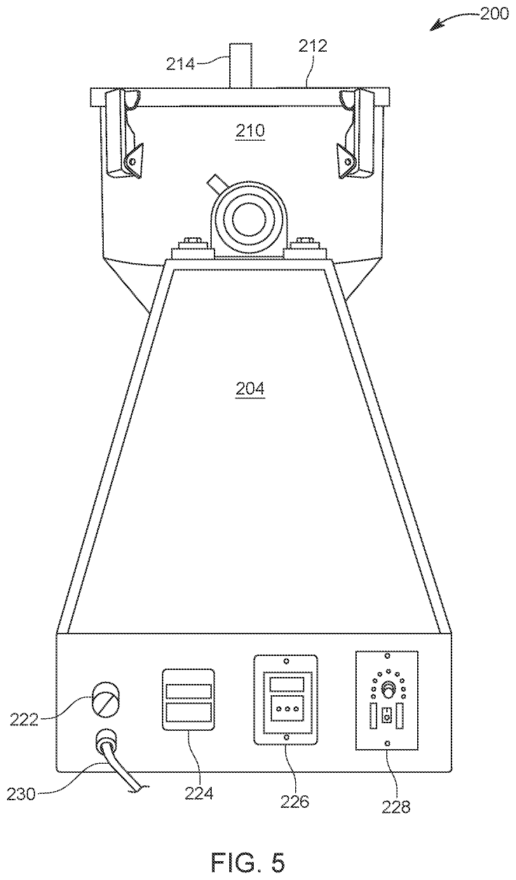

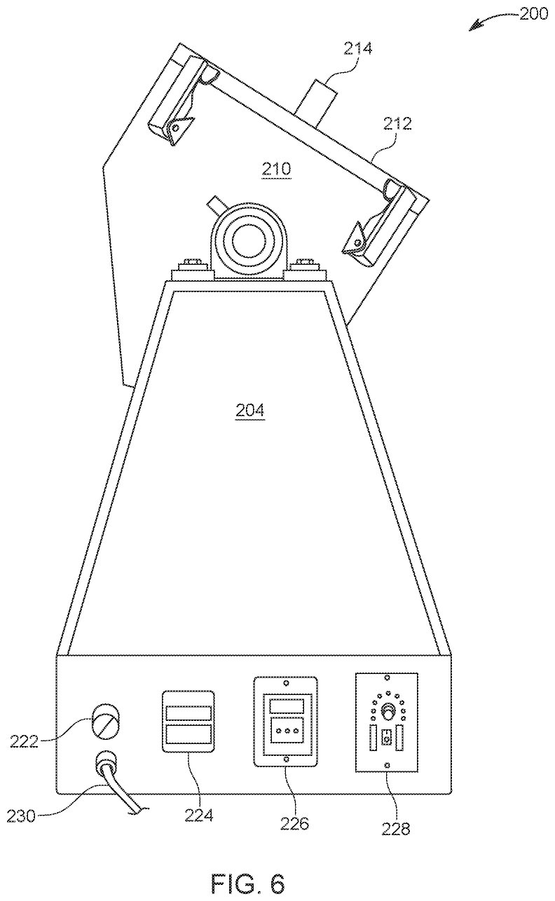

The tumble blender 200 includes an on/off switch 222 (FIGS. 5, 6), a control knob 228, and digital readouts 224, 226 of operational parameters such as revolutions per minute (“RPM”) and a “counter” to keep track of the amount of time the upper and lower sections 210, 218 have been rotating about the horizontal axis X—X. An electrical power source (not shown) provides blender 200 with electrical power via an electrical cord 230.

Tumble blending allows thorough, gentle mixing of the assorted powder ingredient particles without compromising particle integrity. With minimal shear, ingredient particle integrity is maintained, assuring predetermined powder nail polish composition uniformity.

Weight-measured iron oxide powder amounts are added, at step 108, to the tumble blender (see FIG. 1) along with weight-measured amounts of drug and cosmetic (“D&C”) pigment powders, with the predetermined weight-measured amounts of powder-based iron oxides and assortment of predetermined weight-measured amounts of D&C pigment powders added to the tumble blender, depending upon a particular color, or color scheme, preselected for each powder-based nail polish composition of the present subject matter.

A pigment is a colored substance completely or nearly insoluble in water. Dyes, however, are typically water-soluble, at least at some stage in their use. Generally, dyes are often organic compounds, while pigments are often inorganic compounds. Pigments of mixed prehistoric and historic value include ochre, charcoal, and lapis lazuli. Common pigments used in food, drug, and cosmetic (“FD&C”) and/or drug and cosmetic (“D&C”) compositions include: FD&C Blue No. 1 (Aluminum Lake); FD&C Yellow Nos. 5, 6, or 11 (Aluminum Lake); D&C Orange No. 5; D&C Red No. 6 (Disodium Salt or Barium Lake); D&C Red No. 7 (Calcium Lake); and D&C Red Nos. 21, 22, 27, 30, 33, 34, 36, 40. (See U.S. Pat. No. 6,294,013 to Ortlano et al.—hereby incorporated by reference in its entirety.)

Iron oxides are chemical compounds composed of iron and oxygen. Several iron oxides are known. All are black magnetic solids. Often, iron oxides are non-stoichiometric. Oxyhydroxides are a related class of compounds, perhaps the best known of which is rust. Iron oxides and oxyhydroxides are widespread in nature and play an important role in many geological and biological processes. They are used, e.g., as iron ores, pigments, catalysts. Select ones are used in thermite or biologically incorporated into hemoglobin. Iron oxides are inexpensive and are often used as durable pigments in paints, coatings, and colored concretes. Colors commonly available for iron oxides are in an “earthy” end of the yellow/orange/red/brown/black range. Iron oxides are present in ferrous (Fe(II)) or in ferric (Fe(III)) form. They adopt octahedral or tetrahedral coordination geometry. Only a few oxides are significant at the earth's surface, e.g., wüstite, magnetite, and hematite.

Fe(II) oxides of formula FeO include iron(II) oxide and wüstite. Fe(III) oxides of formula Fe₂O₃, iron(III) oxide, include the four known phases: α-Fe₂O₃ (alpha phase, hematite), β-Fe₂O₃ (beta phase), γ-Fe₂O₃ (gamma phase, maghemite), and ε-Fe₂O₃ (epsilon phase). Mixed oxides of Fe(II) and Fe(III) include Fe₃O₄ (iron(II) and iron(III) oxide, magnetite), also with formulas of: Fe₄O₅, Fe₅O₆, Fe₅O₇, Fe₁₃O₁₉, and Fe₂₅O₃₂.

After weight-measured amounts of powder iron oxides and powder D&C pigments have been added to the tumble blender, all powder-based ingredients (at that time) in the tumble blender are tumbled for an amount of time ranging from about 1 minute (“min.”) to about 20 min., or from about 2 min. to about 18 min., or from about 3 min. to about 15 min., or from about 4 min. to about 10 min., or for about 7 min. (See step 110 in FIG. 1.)

The tumbled powder nail polish ingredients are then allowed to remain within the tumble blender for an amount of time ranging from about 1 minute to about 10 minutes, or from about 2 minutes to about 5 min., or for about 3 minutes (See step 112 in FIG. 1.)

Next, at a decision point, step 114, a formulator of this batch of powder nail polish must decide whether the nail polish is to provide nails with a matte finish or whether a shimmery shade is to be selected for a nail polish batch being formulated. Since each batch, at this point, will otherwise result in the nail polish providing a matte finish to nails, if it is decided that a matte finish is desired for the powder nail polish batch, then path 116 is followed, resulting in step 118, which is to open the bottom of the blender and bag the nail polish shade, after which, step 120, signals an end to the manufacture of this batch.

A method for manufacturing a powder-based nail polish composition having a matte finish for application to at least one tacky base coat layer predisposed on at least one associated fingernail and/or on at least one associated toenail of a person includes obtaining predetermined individually weighed amounts of each of these ingredients: kaolin; delaminated talc, uncoated sericite mica; sericite mica coated with carnauba wax; magnesium myristate; micro fine silk powder; and mixed tocopherols. The ingredients are each present as a dry powder. The dry powder ingredients are introduced into a tumble blender. The dry powder ingredients are tumble blended in the tumble blender for a first predetermined amount of time. Next, a predetermined weighed amount of at least one iron oxide of a plurality of iron oxides and a predetermined weighed amount of at least one pigment of a plurality of pigments are added to the tumble blender. The iron oxide and the pigment are each present as a dry powder. Then, the iron oxide, pigment, and dry powder ingredients within the tumble blender are tumble blended for a second predetermined amount of time. The oxide, pigment, and powder ingredients in the blender constitute the powder-based nail polish composition. The powder-based nail polish composition in the tumble blender is caused to remain in the tumble blender for a third predetermined amount of time. Finally, if the tumble blender-contained powder-based nail polish composition is to have a matte finish, the tumble-blended powder-based nail polish composition is removed from the tumble blender and bagged. In embodiments, the first predetermined amount of time is about 5 minutes, the second predetermined amount of time is about 7 minutes, and the third predetermined amount of time is about 3 minutes.

Otherwise, when at decision point, step 114, if this batch of nail polish is to provide a “shimmer” shade to toe and/or fingernails of people, then path 124 is followed, leading to step 126, which is to add weight-measured amounts of mica (in standard white or silver) and/or mica (in various color shades) to the powder ingredients in the tumble blender. Thereafter, step 128, tumbling all of the powder ingredients, now in the tumble blender, for about four minutes. Next, at step 130, the batch would be “held” in the tumble blender for several minutes (e.g., from about five to about twenty minutes). After which, step 118 is repeated, and step 120 is also repeated. It is noted, when mica is added to the blender for a “shimmer” shade, that the mica is combined with the other ingredients in the blender, at a blender rotational speed of about 5 to about 8 RPM, with a 2-3 wt.-% increase or decrease in weight of the nail polish, depending on mica particle size, from fine to large.

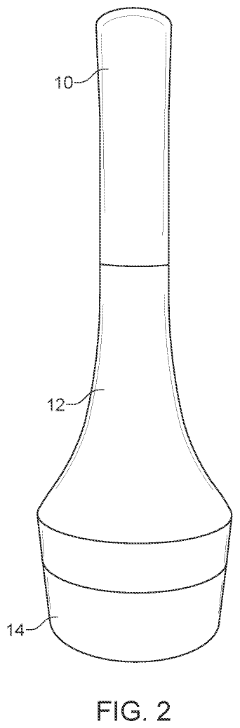

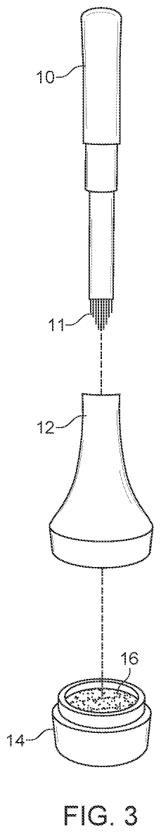

Referring next to FIGS. 2 and 3, the present subject matter may include a toenail or fingernail polish composition 16, taking a powder form and including a plurality of the following ingredients: iron oxide, D&C pigments (such as FDA approved color additives for use in cosmetics), mica, kaolin, sericite mica, magnesium myristate, micro fine silk powder, delaminated talc, and mixed tocopherols, in powder form. The iron oxide and D&C pigments may vary in weight, depending on the polish color. The sericite mica may be uncoated or coated with carnauba wax. The micas may be in white, silver, or various shades of color depending on a desired color for a powder-based nail polish composition.

Referring again to FIG. 1, a method of manufacturing a powder-based fingernail or toenail polish composition 16 (FIG. 3) may include mixing at least one of the iron oxide and D&C pigments together with the kaolin for adhesion, with sericite mica for slip, as well as the magnesium myristate as a non-whitening binder for providing additional adhesion. Delaminated talc may be added for translucency and for maintaining true and bright colors. The micro fine silk powder may be added for a smooth silky texture, while the mixed tocopherols, in powder form, may be mixed in as a preservative fora matte nail polish color. Standard white or silver micas may be added for a shimmer nail polish and other micas various shades of color may be added for shimmery color. Note that the plural ingredients identified above do not include any amount(s) of these following ingredients: a resin, a polymer, an acrylic, a plasticizer, a nail hardening agent, or any liquid adhesive.

Kaolin, sericite mica (coated and uncoated), magnesium myristate, micro fine silk powder, and mixed tocopherols, all in powder form, are “underlying” ingredients of a powder-based nail polish composition of the present subject matter; and combining these powder ingredients requires carefully weighed-amounts of each to achieve desired products and effective blending via gentle tumbling, using, e.g., a commercial tumble blender. Achieving a consistent blend is essential when making a high-quality product for discerning consumers and satisfying regulatory controls. A preferred tumble blender is horizontally mounted and is available in several styles including an IBC (intermediate bulk container) blender, a double-cone configuration, and V-shaped (i.e., frustoconical-shape) configuration. Regardless of the particular geometry, a tumble blender often rotates at an operating speed of from about 3 to about 60 revolutions per minute (“RPM”) or from about 3 to about 50 RPM or from about 3 to about 40 RPM or from about 3 to about 30 RPM or from about 3 to about 20 RPM or from about 3 to about 15 RPM or from about 3 to about 10 RPM or from about 5 to about 25 revolutions per minute (“RPM”), depending upon the ingredients that are to be blended. Tumble blenders are designed to cause a first portion of the powder ingredients to roll, cascade, and fall upon exposed surfaces of a second portion of the powder ingredients being tumbled, while the powder ingredients are caused to rotate about a horizontal axis within blenders for a predetermined time. For V-shaped (i.e., single cone) blenders, all the powder ingredients are continuously split and then gently recombined within the blender. Some tumble blenders include a variable frequency drive, for enabling a gentle, slow start-up when fully loaded, to avoid shearing particles.

In embodiments, the weight percentages for each powder ingredient of the plural “underlying” ingredients are as follows: Amounts of magnesium myristate, relative to all powder-based nail polish composition ingredients, range from about 20 weight-percent (“wt.-%”) to about 50 wt.-%, or from about 20 wt.-% to about 32.5 wt.-%, or from about 32.5 wt.-% to about 50 wt.-%. Amounts of kaolin, in relation to all powder-based nail polish composition ingredients, range from about 10 wt.-% to about 35 wt.-%, or from about 10 wt.-% to about 29 wt.-%, or from about 29 wt.-% to about 35 wt.-%. Amounts of uncoated sericite mica, in relation to all powder-based ingredients in the powder-based nail polish composition, range from about 15 wt.-% to about 40 wt.-%, or from about 15 wt.-% to about 21.6 wt.-%, or from about 21.6 wt.-% to about 40 wt.-%. Amounts of coated (carnauba wax) sericite mica, in relation to all powder nail polish composition ingredients, range from about 5 wt.-% to about 15 wt.-%, or from about 5 wt.-% to about 7.2 wt.-%, or from about 7.2 wt.-% to about 15 wt.-%. Amounts of delaminated talc, in relation to all powder nail polish composition ingredients, range from about 3 wt.-% to about 20 wt.-%, or from about 3 wt.-% to about 5.7 wt.-%, or from about 5.7 wt.-% to about 20 weight-%. Amounts of micro fine silk powder, relative to all powder-based nail polish composition ingredients, range from about 1 wt.-% to about 10 wt.-%, or from about 1 wt.-% to about 3.7 wt.-%, or from about 3.7 wt.-% to about 10 wt.-%. Amounts of tocopherol, in relation to all powder nail polish composition ingredients, range from about 0.1 wt.-% to about 4 wt.-%, from about 0.1 wt.-% to about 0.3 wt.-%, or from about 0.3 wt.-% to about 4 wt.-%.

Amounts of pigment, in relation to powder nail polish composition ingredients, can range from about 3 weight-percent (“wt.-%”) to about 60 wt.-%, or from about 3 wt.-% to about 55 wt.-%, or from about 3 wt.-% to about 50 wt.-%, or from about 3 wt.-% to about 40 wt.-%, from about 3 wt.-% to about 30 wt.-%, or from about 3 wt.-% to about 20 wt.-%, or from about 3 wt.-% to about 15 wt.-%, or from about 3 wt.-% to about 10 wt.-%, or from about 3 wt.-% to about 5 wt.-%, depending on number of shades, used to make a color.

Next, the composition ingredients are set aside when an individual decides to continue formulating the polish composition 16 by weighing predetermined amounts of pigment, such as iron oxide and D&C pigments, varying the weights of the pigment(s) depending on various predetermined nail polish colors, then adding them to the tumble blender. The composition ingredients and various added pigments may next be tumbled for approximately seven minutes. Then, the formulator may allow the batch to sit for about three minutes to allow any dust plumes to dissipate before opening. Next, the formulator may open the output end of the tumble blender and then “bag” the finished matte shade.

To create a shimmer shade, an individual may perform steps described above, and then carefully weigh the micas (in standard white, silver and/or other color shade) and add to the input end of the tumble blender. Tumble for an additional four minutes, allowing the ingredients to sit for a few more minutes to dissipate any dust plume(s). Thereafter, open the output end of the tumble blender and “bag” the nail polish shade.

Before applying a base coat, nails are cleaned with a liquid (e.g., acetone) used to remove all prior polish applications, compositions, oils, and other debris from the nails.

In embodiments, a base coat that remains sticky after LED-light curing is used, for causing a powder-based nail polish composition of the present invention to adhere to it. Any clear base coat containing, e.g., the compound di-HEMA trimethylhexyl dicarbamate, which is an ingredient that can be cured under LED light, would be a suitable base coat.

Methods of applying the powder-based nail polish composition 16 of the present subject matter to the toenails and/or fingernails of oneself or another by brush-painting fingernails with a powdered nail polish composition adapted to only adhere to a base coat, and not the rest of a toe, finger or cuticle, enable the present subject matter to overcome disadvantages of the prior art, by using a procedure which can be finished-and-dried in minutes, for reducing a likelihood of ruining finished applications of toe or fingernail polish.

The user may simply “paint” a fingernail with a clear base coat and then may cure it under LED light for about 30 seconds. The user may next load bristles 11 of a brush 10 with the powder-based composition 16 using the bristles 11 to dab or “brush” the powder composition onto or the clear and tacky base coat, starting from back or front. The user may also “paint” on a clear topcoat requiring LED-curing, to assure that a powder-based polish composition applied to toe or fingernails isn't smudged or discolored. Under LED light (bulbs or lamps) a topcoat will be cured to suitable hardness within about 30 seconds. Since the powder nail polish composition 16 is a dry powder, accidentally smudging or nicking a resulting finished application—i.e., onto a finger and/or toenail—is impossible.

Also, since the powder only adheres to the tacky base coat, no powder will remain on the fingers of one applying the powder fingernail polish composition, resulting in the elimination of clean-up. The customizable powder polish of the present subject matter makes it easier for a user to be creative, and paint multiple colors and/or unique designs.

The ability of a formulator to customize the colors distinguishes the present subject matter from what is currently available in liquid form, where the colors are predetermined.

The powder-based fingernail polish composition of the present subject matter can be stored in a base portion 14 of a bottle 12 (FIGS. 2, 3) adapted to operatively associate with a brush 10 before, during, and after application of the powder nail polish composition 16 to finger and/or toenails of a person. The powder-based composition 16 (FIG. 3) can also be used, e.g., to color such objects as figurines, crafts, and plastic or metallic objects.

Illustrated and described in this patent specification is a powder polish composition and methods of manufacturing and applying the same. The nail polish composition is for application to at least one tacky base coat layer predisposed on at least one associated fingernail and/or at least one associated toenail of a person. The composition includes these ingredients: kaolin; uncoated sericite mica; sericite mica coated with carnauba wax; magnesium myristate; micro fine silk powder; delaminated talc; and mixed tocopherols. Each ingredient is present in the nail polish composition in a dry powder form. The nail polish composition, when applied to at least one tacky base coat layer predisposed on at least one associated fingernail and/or at least one associated toenail of a person, adheres to the at least one tacky base coat layer predisposed on at least one associated fingernail and/or at least one associated toenail of a person. While the present subject matter has been described with reference to certain embodiments, the present subject matter is not limited to these embodiments. On the contrary, many alternatives, changes, and/or modifications shall become apparent to a person of ordinary skill in the art (“POSITA”) after this patent specification and its associated figures have been reviewed. Therefore, alternatives, changes, and modifications are to be treated as forming a part of the present subject matter insofar as they fall within the spirit and scope of the appended claims.

I claim:

1. A dry powder-based nail polish composition for application to at least one tacky base coat layer on at least one fingernail and/or at least one toenail of a person, wherein the composition comprises the following plurality of ingredients:
a kaolin;
an uncoated sericite mica;
a coated sericite mica;
a magnesium myristate;
a micro fine silk powder;
a delaminated talc; and
mixed tocopherols;
wherein each ingredient is present in the nail polish composition as a dry powder and in a predetermined weighed amount relative to other ingredients of the composition; and
wherein the dry powder-based nail polish composition, when applied to at least one tacky base coat layer disposed on at least one fingernail and/or at least one toenail of a person, adheres to the at least one tacky layer on the at least one nail of the person.

2. The dry powder-based nail polish composition of claim 1, further including predetermined weighed amounts of each of the following additional ingredients: a white mica or a silver mica and at least one of a plurality of different color-shaded micas.

3. The nail polish composition of claim 2, further including at least one pigment.

4. The dry powder-based nail polish composition of claim 1, wherein the coated sericite mica ingredient is coated with a predetermined amount of a carnauba wax.

5. The dry powder-based nail polish composition of claim 4, wherein the kaolin constitutes from about 10 weight-percent (“wt.-%”) to about 35 wt.-% of the composition; wherein the uncoated sericite mica constitutes from about 15 wt.-% to about 40 wt.-% of the composition; wherein the coated sericite mica constitutes from about 5 wt.-% to about 15 wt.-% of the composition; wherein the magnesium myristate constitutes from about 20 wt.-% to about 50 wt.-% of the composition; wherein the micro fine silk powder constitutes from about 1 wt.-% to about 10 wt.-% of the composition; wherein the delaminated talc constitutes from about 3 wt.-% to about 20 wt.-% of the composition; and wherein the mixed tocopherols constitute from about 0.1 wt.-% to about 4 wt.-% of the composition.

6. A method of manufacturing a dry powder-based nail polish composition for application to at least one tacky base coat layer disposed on at least one fingernail and/or at least one toenail of a person, wherein the method comprises the following steps:
obtaining predetermined individually weighed amounts of each of the following ingredients: a kaolin; a delaminated talc, an uncoated sericite mica; a sericite mica coated with a predetermined amount of a carnauba wax; a magnesium myristate; a micro fine silk powder; and mixed tocopherols, wherein the ingredients are each present in the nail polish composition as a dry powder;
introducing the dry powder ingredients into a tumble blender and tumble blending the ingredients in the tumble blender for a first predetermined amount of time;
adding a predetermined weighed amount of at least one iron oxide of a plurality of iron oxides and a predetermined weighed amount of at least one pigment of a plurality of pigments to the ingredients in the blender, wherein the at least one iron oxide and the at least one pigment are each present as a dry powder;
tumble blending the at least one iron oxide added to the tumble blender, the at least one pigment added to the tumble blender, and the dry powder ingredients contained in the tumble blender for a second predetermined amount of time, wherein the at least one oxide, the at least one pigment and the dry powder ingredients in the blender constitute the powder-based nail polish composition;
causing the powder-based composition contained in the tumble blender to be held within the tumble blender for a third predetermined amount of time;
if the tumble blender-held powder-based composition is to have a shimmer shade, generating a shimmer shade by adding a predetermined weighed amount of a white mica or a silver mica and a predetermined weighed amount of at least one different color-shaded mica of a plurality of different color-shaded mica, to the powder-based nail polish composition contained within the tumble blender;
tumble blending the tumble blender-contained powder-based nail polish composition for a fourth predetermined amount of time;
causing the tumble blender-contained dry powder-based nail polish composition to remain in the tumble blender for a fifth predetermined amount of time; and
removing the blended powder-based nail polish composition from the blender,
wherein the blended dry powder-based nail polish composition removed from the tumble blender is adapted for application to at least one tacky base coat layer disposed on at least one fingernail and/or at least one toenail of the person.

7. The method of claim 6, wherein the first predetermined amount of time is about 5 minutes, wherein the second predetermined amount of time is about 7 minutes, wherein the third predetermined amount of time is about 3 minutes, and wherein the fourth predetermined amount of time is about 4 minutes.

8. A method of manufacturing a powder-based nail polish composition for application to at least one tacky base coat layer disposed on at least one fingernail and/or on at least one toenail of a person, wherein the method comprises the following steps:
obtaining predetermined individually weighed amounts of each of the following ingredients: a kaolin; a delaminated talc, an uncoated sericite mica; a sericite mica coated with a predetermined amount of a carnauba wax; a magnesium myristate; a micro fine silk powder; and mixed tocopherols, wherein the ingredients are each present in the nail polish composition as a dry powder;
introducing the dry powder ingredients into a tumble blender and tumble blending the ingredients in the tumble blender for a first predetermined amount of time;
adding a predetermined weighed amount of at least one iron oxide of a plurality of iron oxides and a predetermined weighed amount of at least one pigment of a plurality of pigments to the tumble blender, wherein the at least one iron oxide and the at least one pigment are each present as a dry powder;
tumble blending the at least one iron oxide added to the tumble blender, the at least one pigment added to the tumble blender, and the dry powder ingredients contained within the tumble blender for a second predetermined amount of time, wherein the at least one oxide, the at least one pigment and the dry powder ingredients in the tumble blender constitute the powder-based composition;
causing the powder-based composition in the tumble blender to be contained within the tumble blender for a third predetermined amount of time; and
if the tumble blender-contained powder-based composition is to have a matte finish, removing the tumble-blended powder-based composition from the tumble blender,
wherein the blended dry powder-based nail polish composition removed from the tumble blender is adapted for application to at least one tacky base coat layer disposed on at least one fingernail and/or at least one toenail of the person.

9. The method of claim 8, wherein the first predetermined amount of time is about 5 minutes, wherein the second predetermined amount of time is about 7 minutes, and wherein the third predetermined amount of time is about 3 minutes.

10. A method of applying a powder-based nail polish composition to a fingernail and/or to a toenail of a person, wherein the method comprises the following steps:
applying at least one tacky base coat layer to at least one fingernail and/or to at least one toenail of a person;
introducing into a base portion (14) of a bottle (12) a dry powder-based nail polish composition (16) comprising predetermined weight-measured amounts of each of the following ingredients: a kaolin; an uncoated sericite mica; a sericite mica coated with a predetermined amount of a carnauba wax; a delaminated talc; a magnesium myristate; a micro fine silk powder; mixed tocopherols; at least one iron oxide of a plurality of iron oxides; and at least one pigment of a plurality of pigments, wherein each ingredient of the composition is a dry powder;
dipping bristles (11) of a brush (10) of the bottle (12) into the base portion (14) for a predetermined amount of time for causing a portion of the powder composition (16) contained in the base portion (14) to adhere to the brush bristles (11); and
using the brush bristles (11) to apply the powder composition (16) to the tacky layer disposed on the at least one toe or on the fingernail of oneself or another person.

11. The method of claim 10, wherein the kaolin constitutes from about 10 weight-percent (“wt.-%”) to about 35 wt.-% of the composition; wherein the uncoated sericite mica constitutes from about 15 wt.-% to about 40 wt.-% of the composition; wherein the coated sericite mica constitutes from about 5 wt.-% to about 15 wt.-% of the composition; wherein the magnesium myristate constitutes from about 20 wt.-% to about 50 wt.-% of the composition; wherein the micro fine silk powder constitutes from about 1 wt.-% to about 10 wt.-% of the composition; wherein the delaminated talc constitutes from about 3 wt.-% to about 20 wt.-% of the composition; and wherein the mixed tocopherols constitute from about 0.1 wt.-% to about 4 wt.-% of the composition.

12. The method of claim 10, wherein the powder-based nail polish composition further includes: predetermined weighed amounts of a white mica or a silver mica and at least one of a plurality of different color-shaded micas.
